(19) United States
(12) Patent Application Publication
(10) Pub. No.: US 2013/0317325 A1
(43) Pub. Date: Nov. 28, 2013

(54) APPARATUS AND METHOD FOR MEASUREMENT OF PHYSIOLOGICAL PARAMETERS IN TISSUE OF A PATIENT

(71) Applicant: Lockett E. Wood, Lyons, CO (US)

(72) Inventor: Lockett E. Wood, Lyons, CO (US)

(73) Assignee: Nellcor Puritan Bennett LLC, Boulder, CO (US)

(21) Appl. No.: 13/477,388

(22) Filed: May 22, 2012

(51) Int. Cl. A61B 5/1455

(52) U.S. Cl. 600/310

(57) ABSTRACT

A system to optically measure a physiological parameter of tissue of a patient is provided. The system includes a tissue interface assembly configured to emit an optical signal into the tissue, receive a first measurement signal based on the optical signal propagating along a first path, receive a second measurement signal based on the optical signal propagating along a second path, and transfer the first measurement signal and the second measurement signal for delivery to a processing system. The processing system is coupled to the tissue interface assembly and configured to receive the first measurement signal and the second measurement signal, determine a phase delay between the first measurement signal and the second measurement signal based on a cross correlation analysis, and identify a value of the physiological parameter of the patient based on at least the phase delay between the first measurement signal and the second measurement signal.

### TECHNICAL FIELD

Aspects of the disclosure are related to the field of medical devices, and in particular, measuring physiological parameters of blood based on photon density waves emitted into tissue.

### TECHNICAL BACKGROUND

Various devices, such as pulse oximetry devices, can measure some parameters of blood flow in a patient, such as heart rate and oxygen saturation of hemoglobin. Pulse oximetry devices are a non-invasive measurement device, typically employing solid-state lighting elements, such as light-emitting diodes (LEDs) or solid state lasers, to introduce light into the tissue of a patent. The light is then detected and analyzed to determine the parameters of the blood flow in the patient. However, conventional pulse oximetry devices typically only measure certain blood parameters, and are subject to patient-specific noise and inconsistencies which limits the accuracy of such devices.

### OVERVIEW

A system to optically measure a physiological parameter of tissue of a patient is provided. The system includes a tissue interface assembly configured to emit an optical signal into the tissue, receive a first measurement signal based on the optical signal propagating along a first path, receive a second measurement signal based on the optical signal propagating along a second path, and transfer the first measurement signal and the second measurement signal for delivery to a processing system.

The processing system is coupled to the tissue interface assembly and configured to receive the first measurement signal and the second measurement signal, determine a phase delay between the first measurement signal and the second measurement signal based on a cross correlation analysis, and identify a value of the physiological parameter of the patient based on at least the phase delay between the first measurement signal and the second measurement signal.

Another example system to optically measure a physiological parameter of tissue of a patient is provided. The system includes a transmission module, configured to generate an optical signal, and a tissue interface assembly coupled to the transmission module and configured to receive the optical signal, emit the optical signal into the tissue, receive a reference signal based on the optical signal propagating along a first path, receive a measurement signal based on the optical signal propagating along a second path, and transfer the reference signal and the measurement signal for delivery to a receiver module.

The receiver module is coupled to the tissue interface assembly and configured to receive the reference signal and the measurement signal from the tissue interface assembly, convert the reference signal into a digital reference signal, and convert the measurement signal into a digital measurement signal.

The system also includes a back end module coupled to the receiver module and configured to receive the digital reference signal and the digital measurement signal from the receiver module, determine a phase delay between the digital reference signal and the digital measurement signal based on a cross correlation analysis, and identify a value of the physiological parameter of the patient based on at least the phase delay between the digital reference signal and the digital measurement signal.

A method to optically measure a physiological parameter of tissue of a patient is also provided. The method includes emitting an optical signal into the tissue, receiving a first measurement signal based on the optical signal propagating along a first path, and receiving a second measurement signal based on the optical signal propagating along a second path.

The method also includes determining a phase delay between the first measurement signal and the second measurement signal based on a cross correlation analysis, and identifying a value of the physiological parameter of the patient based on at least the phase delay between the first measurement signal and the second measurement signal.

A non-transitory computer-readable medium having instructions stored thereon for analyzing physiological parameters of patients is also provided. The instructions, when executed by a processing system, direct the processing system to determine a phase delay based on a cross correlation analysis between a first measurement signal from an optical signal propagating along a first path through tissue in a patient and a second measurement signal from the optical signal propagating along a second path through the tissue.

The instructions also direct the processing system to identify a value of the physiological parameter of the patient based on at least the phase delay between the first measurement signal and the second measurement signal.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 is a system diagram illustrating a system to optically measure a physiological parameter of tissue of a patient.

FIG. 2 is a flow diagram illustrating a method of operation of a system to optically measure a physiological parameter of tissue of a patient.

FIG. 3 is a system diagram illustrating a system to optically measure a physiological parameter of tissue of a patient.

FIG. 4 is a block diagram illustrating a transmission module and a receiver module within a system to optically measure a physiological parameter of tissue of a patient.

FIG. 5 is a block diagram illustrating a control module, a back end module, and a data processing module within a system to optically measure a physiological parameter of tissue of a patient.

FIG. 6 is a block diagram illustrating a phase processing module within a system to optically measure a physiological parameter of tissue of a patient.

FIG. 7 includes a graph illustrating example parameter measurements.

FIG. 8 includes two graphs illustrating an example phase delay calculation technique using cross correlation analysis.

FIG. 9 includes a graph illustrating example signal to noise ratios.

FIG. 10 is a block diagram illustrating a processing module within a system to optically measure a physiological parameter of tissue of a patient.

### DETAILED DESCRIPTION

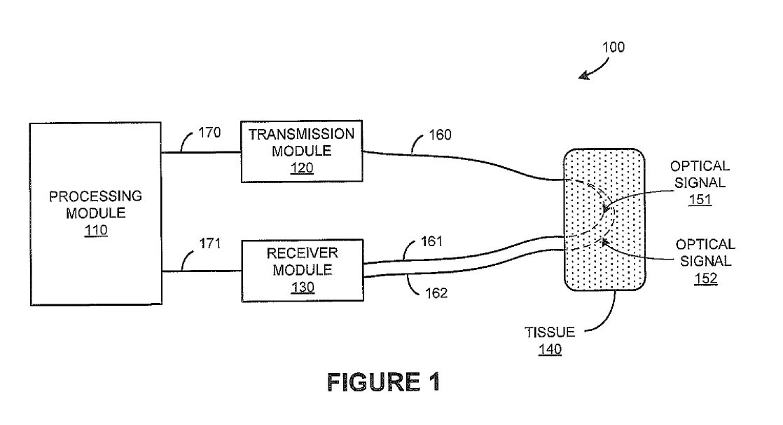

FIG. 1 is a system diagram illustrating system 100 for measuring a physiological parameter of tissue in a patient. FIG. 1 includes processing module 110, transmission module 120, receiver module 130, and tissue 140. Processing module 110 and transmission module 120 communicate over link 170. Processing module 110 and receiver module 130 communicate over link 171. Transmission module 120 emits optical signals over link 160. Receiver module 130 receives optical signals over links 161 and 162. Instructions for operating system 100 may be provided by a non-transitory computer-readable media. In FIG. 1, link 160, link 161, and link 162 are shown located an exemplary distance apart, but can be located on the surface of tissue 140 at predetermined locations or distances, Tissue 140 is a portion of the tissue of a patent undergoing measurement of a physiological blood parameter, and is represented by a rectangular element for simplicity in FIG. 1. Although the term ‘optical’ is used herein for convenience, it should be understood that the measurement signals are not limited to visible light, and can comprise any photonic, electromagnetic, or energy signals, such as visible, infrared, ultraviolet, radio, photoacoustic, or other signals.

Processing module 110 comprises communication interfaces, computer systems, microprocessors, circuitry, non-transient computer-readable media, or other processing devices or software systems, and may be distributed among multiple processing devices. Processing module 110 can be included in the equipment or systems of transmission module 120 or receiver module 130, or can be included in separate equipment or systems. Examples of processing module 110 may also include software such as an operating system, logs, utilities, drivers, databases, data structures, processing algorithms, networking software, and other software stored on a non-transient computer-readable medium.

Transmission module 120 comprises electrical to optical conversion circuitry and equipment, optical modulation equipment, and optical waveguide interface equipment. Transmission module 120 can include DDS components, CD/DVD laser driver components, function generators, oscillators, or other signal generation components, filters, delay elements, signal conditioning components, such as passive signal conditioning devices, attenuators, filters, and directional couplers, active signal conditioning devices, amplifiers, or frequency converters, including combinations thereof. Transmission module 120 can also include switching, multiplexing, or buffering circuitry, such as solid-state switches, RF switches, diodes, or other solid state devices. Transmission module 120 also includes laser elements such as a laser diode, solid-state laser, or other laser device, along with associated driving circuitry. Optical couplers, cabling, or attachments can be included to optically mate laser elements to link 160.

Receiver module 130 comprises light detection equipment, optical to electrical conversion circuitry, photon density wave characteristic detection equipment, and analog-to-digital conversion equipment. Receiver module 130 can include a photodiode, phototransistor, avalanche photodiode (APD), photomultiplier, or other optoelectronic sensor, along with associated receiver circuitry such as amplifiers or filters. In some examples, receiver module 130 comprises photoacoustic detection circuitry. Optical couplers, cabling, or attachments can be included to optically mate receiver module 130 to link 161. Receiver module 130 can also include phase and amplitude detection circuitry and processing elements.

In this example embodiment, optical signal 151 follows a first path through tissue 140 resulting in a first measurement signal carried by link 161 to receiver module 130. Also, optical signal 152 follows a second path through tissue 140 resulting in a second measurement signal carried by link 162 to receiver module 130. As illustrated, optical signals 151 and 152 are shown following exemplary paths through tissue 140. These paths are simplified for purposes of clarity, and in reality may differ from the paths illustrated in FIG. 1.

Tissue 140 is a portion of the tissue of a patent undergoing measurement of a physiological blood parameter. It should be understood that tissue 140 can represent a finger, fingertip, toe, earlobe, forehead, or other tissue portion of a patient undergoing physiological parameter measurement. Tissue 140 can comprise muscle, fat, blood, vessels, or other tissue components. The blood portion of tissue 140 can include tissue diffuse blood and arterial or venous blood. In some examples, tissue 140 is a test sample or representative material for calibration or testing of system 100.

Optical links 160-162 each comprise an optical waveguide, and use glass, polymer, air, space, or some other material as the transport media for transmission of light, and can each include multimode fiber (MMF) or single mode fiber (SMF) materials. A sheath or loom can be employed to bundle each of optical links 160-162 together for convenience. One end of each of optical links 160-162 mates with an associated component of system 100, and the other end of each of optical links 160-162 is configured to emit optical signals into tissue 140 or receive optical signals from tissue 140.

Links 170-171 each use metal, glass, optical, air, space, or some other material as the transport media, and comprise analog, digital, RF, optical, or power signals, including combinations thereof. Links 170-171 can each use various communication protocols or formats, such as Controller Area Network (CAN) bus, Inter-Integrated Circuit (I2C), 1-Wire, Radio Frequency Identification (RFID), optical, circuit-switched, Internet Protocol (IP), Ethernet, wireless, Bluetooth, communication signaling, or some other communication format, including combinations, improvements, or variations thereof. Links 170-171 can each be direct links or may include intermediate networks, systems, or devices, and can each include a logical network link transported over multiple physical links.

Communication links 160-162 and 170-171 may each include many different signals sharing the same associated link, as represented by the associated lines in FIG. 1, comprising channels, forward links, reverse links, user communications, overhead communications, frequencies, wavelengths, carriers, timeslots, spreading codes, logical transportation links, packets, or communication directions,

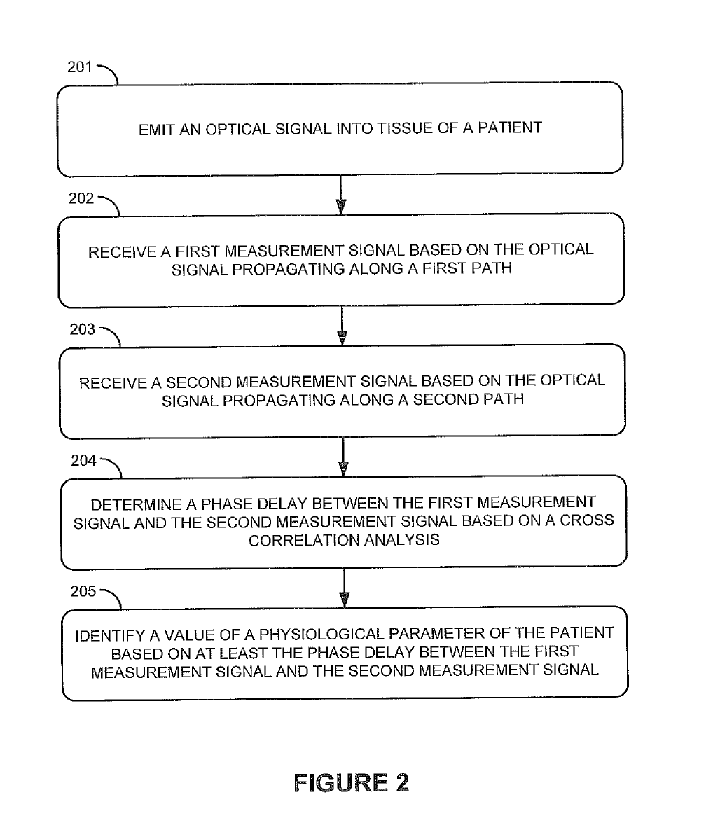

FIG. 2 is a flow diagram illustrating a method of operating system 100 for measuring a physiological parameter of tissue in a patient. The operations of FIG. 2 are referenced herein parenthetically. In FIG. 2, transmission module 120 emits an optical signal into tissue 140 of a patient from a plurality of modulated light sources, (operation 201). In this example, transmission module 120 emits a plurality of photon density waves over link 160 into tissue 140. The plurality of photon density waves emitted into tissue 140 each comprise modulated optical signals, such as modulated laser light. In some examples, each of the plurality of photon density waves comprises at least an individual wavelength of modulated light. Transmission module 120 can receive instructions from processing module 110 regarding the plurality of photon density waves over link 170, among other instructions.

Receiver module 130 receives a first measurement signal through link 161 based on the optical signal 160 propagating along a first path 151 through tissue 140, (operation 202). Receiver module 130 also receives a second measurement signal through link 162 based on the optical signal 160 propagating along a second path 152 through tissue 140, (operation 203). In this example, receiver module 130 can detect the plurality of optical signals (or photon density waves) over links 161 and 162 which were emitted into tissue 140 by transmission module 120. Receiver module 130 detects the plurality of photon density waves in tissue 140 as modulated optical signals. Receiver module 130 typically detects the characteristics of the plurality of photon density waves after being scattered, absorbed, propagated, or transmitted by tissue 140. The characteristics can include amplitude, phase delay, noise, modulations, or other characteristics of each of the plurality of photon density waves. Receiver module 130 then transfers information about the characteristics of the plurality of photon density waves over link 171 to processing module 110.

Processing module 110 determines a phase delay between the first measurement signal and the second measurement signal based on a cross correlation analysis of the two measurement signals, (operation 204). Cross correlation is a measure of the similarity of two waveforms as a function of a time lag applied to one of the waveforms. This is also referred to as a sliding dot product or sliding inner product. Processing module 110 then identifies a value of a physiological parameter of the patient based on at least the phase delay between the first measurement signal and the second measurement signal, (operation 205).

In some embodiments, processing module 110 may be instantiated as a general-purpose processing system receiving instructions in the form of a non-transitory computer-readable medium. In an example embodiment, the medium may contain instructions for analyzing physiological parameters of patients.

In this example, when the instructions are executed by a processing system, they direct the processing system to determine a phase delay based on a cross correlation analysis between a first measurement signal from an optical signal propagating along a first path through tissue in a patient and a second measurement signal from the optical signal propagating along a second path through the tissue. The instructions further direct the processing system to identify a value of the physiological parameter of the patient based on at least the phase delay between the first measurement signal and the second measurement signal.

The physiological parameter can include any parameter associated with blood or tissue 140 of the patient, such as total hemoglobin concentration (tHb), regional oxygen saturation (rSO2), or arterial oxygen saturation (SpO2), among other parameters, including combinations thereof. The characteristics of the plurality of optical signals (or photon density waves) can change during pulsatile perturbation of tissue 140. These changing photon density wave characteristics are processed along with the pulsatile perturbation characteristics to determine a value of the physiological parameter based at least on the phase delay between the first measurement signal and the second measurement signal.

In typical examples, the pulsatile perturbation introduces dynamic, quasi-periodic, or “AC” information into the characteristics of the plurality of photon density waves, and the dynamic characteristics can be processed to determine a value of the physiological parameter. For example, the pulsatile perturbation characteristics can provide an AC amplitude and AC phase delay for each of the plurality of photon density waves, which are then processed to determine a value of the physiological parameter. Time-averaged characteristics, such as “DC” information, can also be taken into account. In some examples, the AC amplitude and AC phase delay can be determined by determining multiple measurements of the amplitude and phase delay over the pulsatile perturbation, and determining differential values of each of the amplitude and phase delay based on the multiple measurements. A ratio of the differential values can then be processed to determine the value of the physiological parameter. The multiple measurements can be taken at similar points during a periodic pulsatile perturbation, such as during subsequent minimal perfusion or blood flow rate times. The multiple measurements can be taken continuously during the pulsatile perturbation, or at varying points during a periodic pulsatile perturbation, such as at maximum perfusion and minimum perfusion times. It should be understood that the terms “AC” and “DC” used herein are not necessarily referring to alternating or direct “currents,” but are instead used to refer to dynamic signal properties for “AC” and relatively stable signal properties for “DC.”

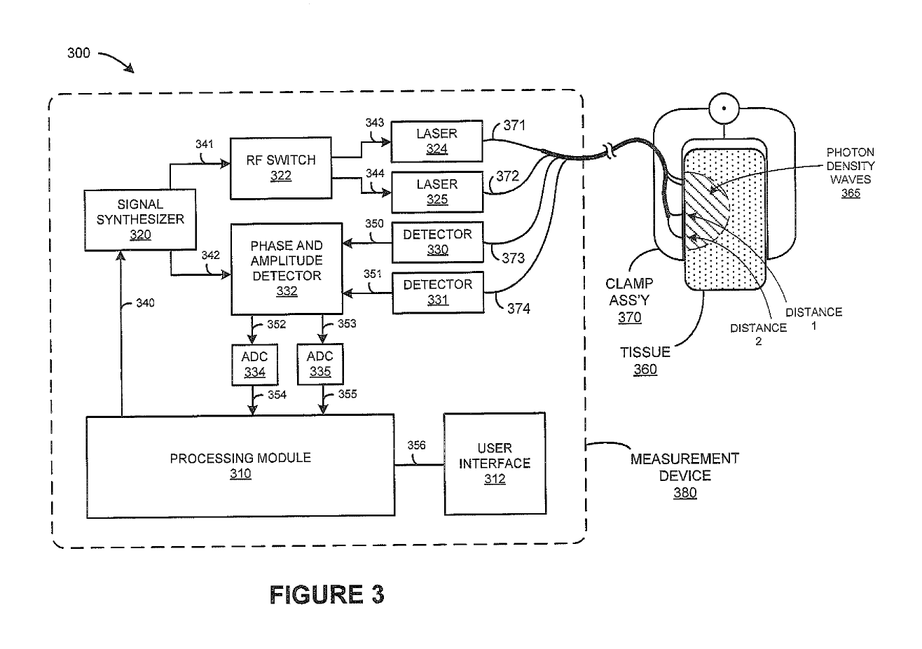

FIG. 3 is a system diagram illustrating system 300 for measuring a physiological parameter of tissue in a patient. System 300 includes tissue 360, clamp assembly 370 (also known as a tissue interface assembly), and measurement device 380. Measurement device 380, in conjunction with clamp assembly 370 is one example embodiment of a system for measuring a physiological parameter of tissue in tissue 360 of a patient. Tissue 360 is a portion of the tissue of a patent undergoing measurement of a physiological tissue parameter, and is represented by a rectangular element for simplicity in FIG. 3. It should be understood that tissue 360 can represent a finger, fingertip, toe, earlobe, forehead, or other tissue portion of a patient undergoing physiological parameter measurement. Tissue 360 can comprise muscle, fat, blood, vessels, or other tissue components. The blood portion of tissue 360 can include tissue diffused blood and arterial or venous blood.

Clamp assembly 370 (also known as a tissue interface assembly) includes a clamp portion and an optical signaling portion. The clamp portion is configured to compressively clamp over a portion of tissue 360 to provide optical mating between ends of optical fibers 371-374 and tissue 360, and can comprise metal, plastic, or composite materials to form the clamp jaw portion. A spring hinge or other element can provide the compressive force to hold clamp assembly 370 onto tissue 360. Other configurations can be employed to provide optical contact between ends of optical fibers 371-374 and tissue 360, such as adhesive pads. Clamp assembly 370 also includes an optical signaling portion which includes optical fibers 371-374. A sheath or loom can be employed to bundle each of optical fibers 371-374 together for convenience. One end of each of optical fibers 371-374 mates with an associated component of measurement device 380, and the other end of each of optical fibers 371-374 is configured to emit light into tissue 360 or receive light from tissue 360. Optical fibers 371-374 each comprise an optical waveguide, such as a glass or polymer fiber, for transmission of light therein, and can include multimode fiber (MMF) or single mode fiber (SMF) materials.

In FIG. 3, optical fibers 371-374 are bundled into a group at measurement device 380, and broken apart into separate fibers at clamp assembly 370. The order/numbering of the optical fiber breakout is the same as that shown for the bundling, i.e. 371 is on ‘top’ and 374 is on ‘bottom’ of FIG. 3. Also shown in FIG. 3 are different distances or spacings for each of the receiving optical fibers 373-374, as compared to the emission fibers 371-372. The distances are indicated by ‘distance 1’ and ‘distance 2’ in FIG. 3. These distances can be determined based on the parameters or characteristics of the tissue or blood are to be monitored, or upon the differences in signal detection at the two distances. For example, ‘distance 1’ can be 7 millimeters, and ‘distance 2’ can be 10 millimeters, although other distances can be used. In FIG. 3, the emission fibers 371-372 as shown to be closely spaced, and can be considered to be at the same contact point on tissue 360, possibly aligned along a spatial dimension protruding from FIG. 3. Also, the configuration of clamp assembly 370 shown in FIG. 3 is for a reflectance-based measurement, where emit and receive fibers are coupled to the same side of tissue 360. In other examples, a transmission-based measurement can be employed, where emit and receive fibers are on opposite sides of tissue 360. A combination of reflectance and transmission can be employed.

Measurement device 380 includes processing module 310, user interface 312, signal synthesizer 320, radio frequency (RF) switch 322, lasers 324-325, detectors 330-331, phase and amplitude (PA) detector 332, and analog-to-digital converters (ADC) 334-335. Processing module 310 and signal synthesizer 320 communicate over link 340. Processing module 310 and ADC 334-335 communicate over associated links 354-355. Processing module 310 and user interface 312 communicate over link 356. Signal synthesizer 320 and RF switch 322 communicate over link 341. Signal synthesizer 320 and PA detector 332 communicate over link 342. RF switch 322 and lasers 324-325 communicate over associated links 343-344. PA detector 332 and ADC 334-335 communicate over associated links 352-353. PA detector 332 and detectors 330-331 communicate over associated links 350-351.

In FIG. 3, links 340-344 and 350-356 each use metal, glass, optical, air, space, or some other material as the transport media, and comprise analog, digital, RF, optical, or power signals, including combinations thereof. Links 340-344 and 350-356 can each use various communication protocols or formats, such as Controller Area Network (CAN) bus, Inter-Integrated Circuit (I2C), 1-Wire, Radio Frequency Identification (RFID), optical, circuit-switched, Internet Protocol (IP), Ethernet, Wireless Fidelity (WiFi), Bluetooth, communication signaling, or some other communication format, including combinations, improvements, or variations thereof. Links 340-344 and 350-356 can each be direct links or may include intermediate networks, systems, or devices, and can each include a logical link transported over multiple physical links.

Although various elements of system 300 are shown in FIG. 3 as included in measurement device 380, as indicated by the dashed box surrounding elements 310, 312, 320, 322, 324-325, 330-331, 332, 334-334, and the associated links, it should be understood other configurations can be employed. Also, the directional arrows shown for the interconnecting links in measurement device 380 are merely used to show an example operational flow, and are not intended to represent one-way communications.

Processing module 310 retrieves and executes software or other instructions to direct the operations of signal synthesizer 320 as well as process data received from ADC 334-335. In this example, processing module 310 comprises a digital signal processor (DSP), and can include a non-transitory computer-readable medium such as a disk, integrated circuit, server, flash memory, or some other memory device, and also may be distributed among multiple memory devices. Examples of processing system 310 include DSPs, micro-controllers, field programmable gate arrays (FPGA), or discrete logic, including combinations thereof. In one example, the DSP comprises an Analog Devices Blackfin® device.

User interface 312 includes equipment and circuitry to communicate information to a user of measurement device 380. User interface 312 may include any combination of displays and user-accessible controls and may be part of system 300 as shown or can be a separate patient monitor or multi-parameter monitor. When user interface 312 is a separate unit, user interface 312 may include a processing system and communication link 356 may be any suitable link for external communication such as a serial port, UART, USB, Ethernet, or wireless link such as Bluetooth, Zigbee or WiFI, among other link types. Examples of the equipment to communicate information to the user can include displays, indicator lights, lamps, light-emitting diodes, haptic feedback devices, audible signal transducers, speakers, buzzers, alarms, vibration devices, or other indicator equipment, including combinations thereof. The information can include raw ADC samples, calculated phase and amplitude information for one or more emitter/detector pairs, blood parameter information, waveforms, summarized blood parameter information, graphs, charts, processing status, or other information. User interface 312 also includes equipment and circuitry for receiving user input and control, such as for beginning, halting, or changing a measurement process or a calibration process. Examples of the equipment and circuitry for receiving user input and control include push buttons, touch screens, selection knobs, dials, switches, actuators, keys, keyboards, pointer devices, microphones, transducers, potentiometers, non-contact sensing circuitry, or other human-interface equipment.

Signal synthesizer 320 generates modulation signals and reference signals for use by other elements of measurement device 380, as well as receives instructions from processing module 310 for generating these signals. In this example, signal synthesizer 320 comprises a two-channel direct digital synthesis (DDS) component, such as Analog Devices AD9958. Signal synthesizer 320 digitally synthesizes drive signal 341 and reference signal 342 at a matched predetermined frequency and waveform, such as a 400 MHz sine wave, although other waveforms and frequencies can be employed. In some examples, a filtered output signal can be used or higher-frequency images of the output frequency can be isolated by filters and used to generate drive signal 341 or reference signal 342. Drive signal 341 and reference signal 342 are precisely synthesized with predetermined amplitude and phase delay relationships to each other. The amplitude of drive signal 341 is synthesized based on the input parameters for lasers 324-325, possibly after further amplification, switching, or multiplexing by RF switch 322. The amplitude of reference signal 342 is synthesized based on the input parameters of PA detector 332, although in some examples levels may be adjusted for optimal power consumption, dynamic range, signal-to-noise ratio at the receiver, or the operating characteristics of PA detector 332. The relative phase between drive signal 341 and reference signal 342 are synthesized based on the phase delay experienced by drive signal 341 through lasers 324-325, optical fibers 371-372, tissue 360, optical fibers 373-374, and detector 330-331, among other factors, such as operating parameters of PA detector 332. For example, the phase delay synthesized by signal synthesizer 320 may be calibrated to achieve a predetermined relative phase delay at PA detector 332, based in part on the length of optical fibers 371-374. Other examples of signal synthesizer 320 include multiple DDS components, CD/DVD laser driver components, such as National Semiconductor LMH6525, function generators, or other signal generation components. However, in examples of CD/DVD laser driver components, additional circuitry may be needed to achieve the precise predetermined amplitude and phase delay relationship between drive signal 341 and reference signal 342, such as filters, delay elements, or other calibration components.

RF switch 322 comprises switching, multiplexing, or buffering circuitry for selectively providing drive signal 341 over any of links 343-344. In this example, RF switch 322 comprises a single-pole double-throw (SPDT) style of switch operable at the high frequencies of drive signal 341 to alternately provide drive signal 341 to either of links 343-344 in a repeating, sequential manner. RF switch 322 can comprise a solid-state switch, such as transistors, RF junctions, diodes, or other solid state devices. In some examples, RF switch 322 receives switching instructions from processing module 310, while in other examples, a predetermined switching profile is included in RF switch 322. In further examples, RF switch 322 includes signal conditioning components, such as passive signal conditioning devices, attenuators, filters, and directional couplers, active signal conditioning devices, amplifiers, or frequency converters, including combinations thereof. In yet further examples, RF switch 322 provides drive signal 341 to both of links 343-344 in a simultaneous manner. In all configurations of RF switch 322, an “off” condition can be employed where drive signal 341 is not provided over any of links 343-344, and links 343-344 can be driven to a predetermined signal state, such as a zero signal level, predetermined DC signal level, or floating-state, among other configurations.

Lasers 324-325 each comprise a laser element such as a laser diode, solid-state laser, or other laser device, along with associated driving circuitry. Lasers 324-325 emit coherent light over associated optical fibers 371-372. In this example, a single wavelength of light is associated with each of lasers 324-325 and likewise each of optical fibers 371-372, and the wavelengths may be of different wavelengths, such as 660 nm for laser 324 and 808 nm for laser 325, although other wavelengths can be used. Each of lasers 324-325 modulate the associated laser light based on an input modulation signal, namely the associated modulation signal received over links 343-344. Optical couplers, cabling, or attachments can be included to optically mate lasers 324-325 to optical fibers 371-372. Additionally, a bias signal may be added or mixed into the modulation signals received over links 343-344, such as adding a “DC” bias for the laser light generation components. In some examples, the bias is adjusted so that the minimum signal level provided to the laser components is at the lasing threshold, or slightly above the lasing threshold.

Detectors 330-331 each comprise a light detector element, such as a photodiode, phototransistor, avalanche photodiode (APD), photomultiplier tube, charge coupled device (CCD), or other optoelectronic sensor, along with associated receiver circuitry such as amplifiers or filters. Detectors 330-331 receive light over associated optical fibers 373-374, and transfer electrical signals over links 350-351. Optical couplers, cabling, or attachments can be included to optically mate detectors 330-331 to optical fibers 373-374. Detectors 330-331 convert the optical signals received over optical fibers 373-374 to electrical signals for transfer over links 350-351. Detectors 330-331 can also include circuitry to condition or filter the signals before transfer over links 350-351. It should be noted that in this example output optical fibers 371-372 each only carry a particular wavelength of light, while input optical fibers 373-374 can carry any received light from tissue 360, which can include multiple wavelengths on each of optical fibers 373-374. Also, although two detectors are shown in FIG. 3, in other examples, a single detector can be shared between multiple laser sources, such as when the detector employs TDM, FDM, CDM, or WDM techniques to detect multiple PDW signals from a combined detected light.

Phase and amplitude (PA) detector 332 comprises circuitry and processing elements to determine amplitudes of signals received over links 350-351, and to determine phase delays of signals received over links 350-351 relative to reference signal 342. In some examples, PA detector 332 comprises a device, such as Analog Devices AD8302, although discrete circuitry can be employed. PA detector 332 provides the amplitude and phase information in an analog format over links 352-353. The phase and amplitude outputs of PA detector 332 can be amplified or conditioned to satisfy the input parameters of ADC 334-335.

ADC 334-335 each comprise analog-to-digital converters. ADC 334-335 receive the amplitude and phase information over associated links 352-353 from PA detector 332, and digitize the amplitude and phase information. The dynamic range, bit depth, and sampling rate of ADC 334-335 can be selected based on the signal parameters of the amplitude and phase information, such as to prevent aliasing, clipping, and for reduction in digitization noise. ADC 334-335 can each be a dual-channel ADC, or be implemented in discrete components. ADC 334-335 provides digitized forms of the amplitude and phase information over links 354-355 for receipt by processing system 310.

Although lasers 324-325 and detectors 330-331 are included in measurement device 380 in FIG. 3, in other examples, lasers 324-325 or detectors 330-331 can be included in clamp assembly 370. Shorter optical fibers 371-374 or other waveguides can be employed when lasers 324-325 or detectors 330-331 are integrated into clamp assembly 370. In some examples, optical fibers 371-374 are not employed between lasers 324-325 and tissue 360, and the laser light is introduced directly into tissue 360, possibly after associated lenses or tissue interface optics. Furthermore, electrical or RF signaling can be employed between clamp assembly 370 and measurement device 380 to drive or receive signals from lasers 324-325 or detectors 330-331.

Although two lasers are shown in FIG. 3 to drive two optical fibers, in other examples, a single optical fiber is employed and a single laser is employed. Also, a greater number of laser sources and detectors can be employed, such as four lasers or four detectors. In further examples, multiple light sources or input fibers can be employed to emit PDWs into tissue 360, but be positioned on tissue 360 at different distances from a common detector or common detection fiber, where multiple input fibers and a single detector or detection fiber can be employed. Likewise, in other examples, a single light source or input fiber can be employed to emit a PDW or PDWs into tissue 360, while multiple detectors or detection fibers are positioned at different distances along tissue 360 from the input.

In yet further examples, the laser light from each of lasers 324-325 is multiplexed or combined onto a single optical fiber. In examples using a single optical fiber to carry multiple optical signals, the multiplexed signals can be time-division multiplexed (TDM), such as when RF switch 322 alternately provides the modulation signal to lasers 324-325, or wavelength-division multiplexed (WDM), such as when RF switch 322 simultaneously provides the modulation signal to lasers 324-325. Frequency-division multiplexing (FDM) can also be employed, where different modulation frequencies are used for each of lasers 324-325. The PDW signals from both lasers can be mixed or combined onto a single optical fiber for emission into tissue 360. Detectors 330-331 can also share a single optical fiber, and perform frequency separation to distinguish the different modulation frequencies of the PDW signals. A single detector can also be employed to detect multiple PDW signals, or to share multiple detection optical fibers. Two optical fibers can also be employed for detection of the PDW signals at different distances on tissue 360, and frequency separation can be performed in each of detectors 330-331 to determine PDW signals for each modulation frequency. Other configurations can be employed, such as code-division multiplexing (CDM), where additional code-based modulation on the optical signals is employed to create code-separated channels. Frequency hopping, chirping, or spread spectrum techniques can also be employed.

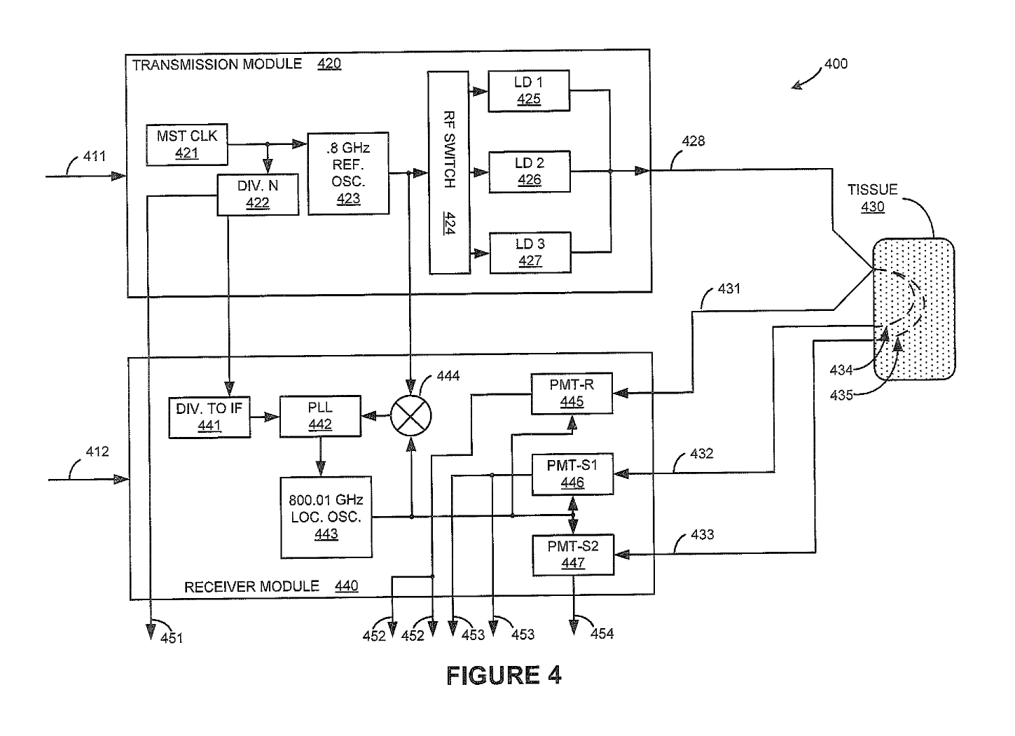

FIG. 4 is a block diagram illustrating an example embodiment of transmission module 420 and receiver module 440 within a system 400 to optically measure a physiological parameter of tissue of a patient. Transmission module 420 is similar in operation to transmission module 120 of FIG. 1, and receiver module 440 is similar in operation to receiver module 130 of FIG. 1, although other configurations can be employed.

Transmission module 420 receives control signals 411 from processing module 110 and outputs optical signal 428 to tissue 430. Receiver module 440 receives control signals 412 from processing module 110 and also receives reference measurement signal 431, first measurement signal 432, and second measurement signal 433 from tissue 430. Reference measurement signal 431 results from optical signal 428 propagating a reference path. The reference path can include an optical shunt from optical signal 428 to reference measurement signal 431, or can include a short distance of propagation of optical signal 428 through tissue 430. First measurement signal 432 results from optical signal 428 propagating along first path 434 through tissue 430. Second measurement signal 433 results from optical signal 428 propagating along second path 435 through tissue 430. Measurement paths 434 and 435 illustrated in FIG. 4 are exemplary paths only and are simplified for clarity purposes.

Transmission module 420 comprises master clock 421, divide-by-N module 422, 0.8 GEN reference oscillator 423, RF switch 424, first laser diode 425, second laser diode 426, and third laser diode 427. The outputs of the three laser diodes 425-427 are combined in any of a wide variety of ways to form optical signal 428. Optical signal 428 is transmitted to tissue 430 where it propagates along various paths associated with tissue 430 resulting in reference signal 431, first measurement signal 432, and second measurement signal 433.

Receiver module 440 comprises divide-to-intermediate frequency module 441, phase locked loop 442, 800.01 GHz local oscillator 443, mixer 444, reference photo-multiplier tube 445, first photo-multiplier tube 446, and second photo-multiplier tube 447. The three photo-multiplier tubes 445-447 are clocked by local oscillator 443. Reference measurement signal 431 is received by photo-multiplier tube 445, first measurement signal 432 is received by photo-multiplier tube 446, and second measurement signal 433 is received by photo-multiplier tube 447. Note that reference measurement signal 431 may include any combination of radio frequency, low frequency, and intermediate frequency signals in some embodiments.

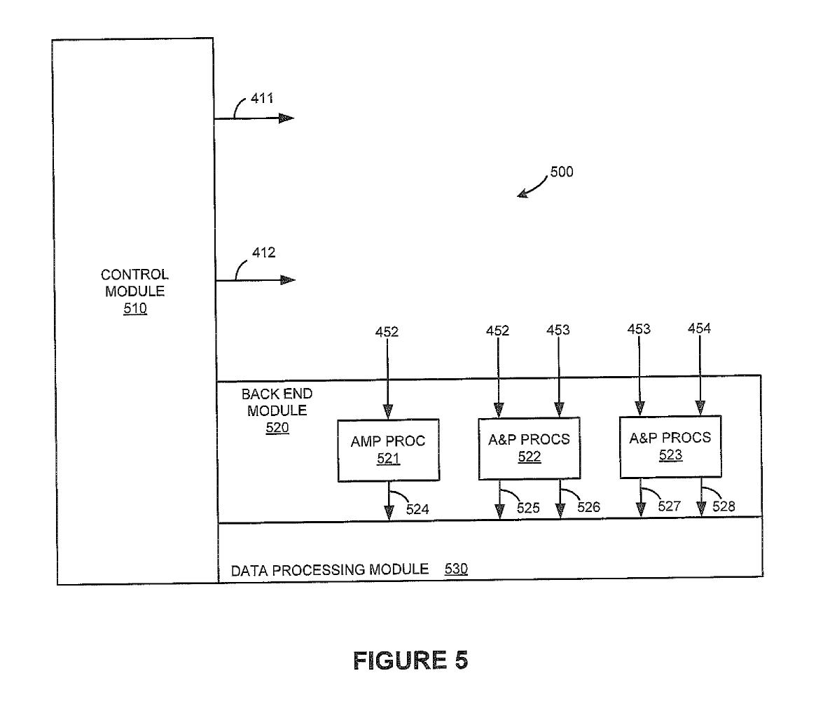

The electrical outputs of the three photo-multiplier tubes 445-447 are provided to a back end module illustrated by example in FIG. 5. Reference photo-multiplier tube 445 provides reference signal 452 to the back end module, while first photo-multiplier tube 446 provides first measurement signal 453 to the back end module, and second photo-multiplier tube 447 provides second measurement signal 454 to the back end module. Receiver module 440 also passes the output of the divide-by-N module 422 from transmission module 420 to the back end module. These signals are used by the back end module and a data processing module to determine the amplitude and phase differences of the first and second measurement signals 432 and 433. The back end module is illustrated in farther detail in FIG. 5. Note that some outputs from receiver module 440 are provided in duplicate to the back end module. Reasons for this will become apparent with examination of back end module 520 in FIG. 5. Back end module 520 includes one amplitude processor and two phase and amplitude processors. Duplicate signals are provided on the output of receiver module 440 to correspond to the inputs required by the processors within back end module 520. While this example embodiment includes one amplitude processor and two phase and amplitude processors, other embodiments may advantageously use other quantities and configurations of these processors. In some embodiments, additional phase processors may be advantageous.

FIG. 5 is a block diagram illustrating a control module 510, a back end module 520, and a data processing module 530 within a system 500 to optically measure a physiological parameter of tissue of a patient. In this example embodiment, several modules of measurement device 380 are illustrated in more detail. Back end module 520 includes modules which correspond in part to phase and amplitude detector 332 illustrated in FIG. 3.

Data processing module 530, control module 510, and back end module 520 are illustrated as exemplary components of processing module 310 from FIG. 3 in this example configuration, however other configurations are also valid. Data processing module 530 receives outputs 524-528 from back end module 520, and processes these outputs 524-528 to identify a value of a physiological parameter of a patient based on at least the phase delay between first measurement signal 432 and second measurement signal 433. Control module 510 generates control signals 411 provided to transmission module 420, and control signals 412 provided to receiver module 440.

Back end module 520 includes amplitude processor 521, amplitude and phase processors 522, and amplitude and phase processors 523. Amplitude processor 521 is configured to receive the reference signal 452 from receiver module 440 and to determine the amplitude 524 of the reference signal 452. Amplitude and phase processors 522 are configured to receive the reference signal 452 and the first measurement signal 453 from receiver module 440 and to determine the amplitude 525 of the first measurement signal 453 and the phase difference 526 between the reference signal 452 and the first measurement signal 453. Amplitude and phase processors 523 are configured to receive the first 10 kHz measurement signal 453 and the second measurement signal 454 from receiver module 440 and to determine the amplitude 527 of the second measurement signal 454 and the phase difference 528 between the first measurement signal 453 and the second measurement signal 454.

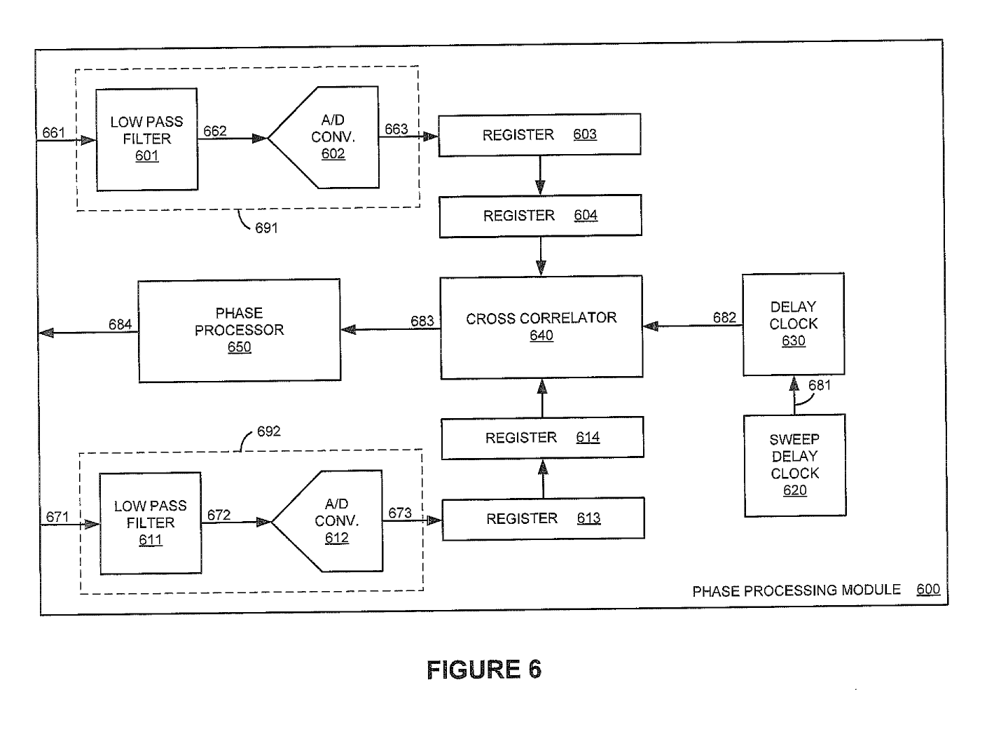

Amplitudes are determined by processors 521-523 in any of a wide variety of standard methods well known to those of skill in the art. Phase differences are determined by phase processors using a cross correlation method, An example phase processing module is illustrated in more detail in FIG. 6.

FIG. 6 is a block diagram illustrating a phase processing module 600 within a system to optically measure a physiological parameter of tissue of a patient. In this example embodiment phase processing module 600 uses cross correlator 640 to generate a plurality of cross coordination coefficients 683 which are used by phase processor 650 to determine a phase difference 684 between a first input signal 661 and a second input signal 671.

Phase processing module 600 receives two inputs 661 and 671 from receiver module 440 and, from those inputs, determines a phase difference 684 between the two inputs 661 and 671. The first input 661 passes through low pass filter 601 producing filtered input 662, which then is processed by analog-to-digital converter 602 producing first digital input 663. First digital input 663 is passed through registers 603 and 604 before reaching cross correlator 640. The second input 671 passes through low pass filter 611 producing filtered input 672, which then is processed by analog-to-digital converter 612 producing first digital input 673. First digital input 673 is passed through registers 613 and 614 before reaching cross correlator 640.

In some embodiments filter/A/D modules 691 and 692 may be provided within receiver module 440. This architecture may be advantageous since it contains all of the analog circuitry within transmission module 420 and receiver module 440, and thus processing module 310, and back end module 520 contain purely digital circuitry.

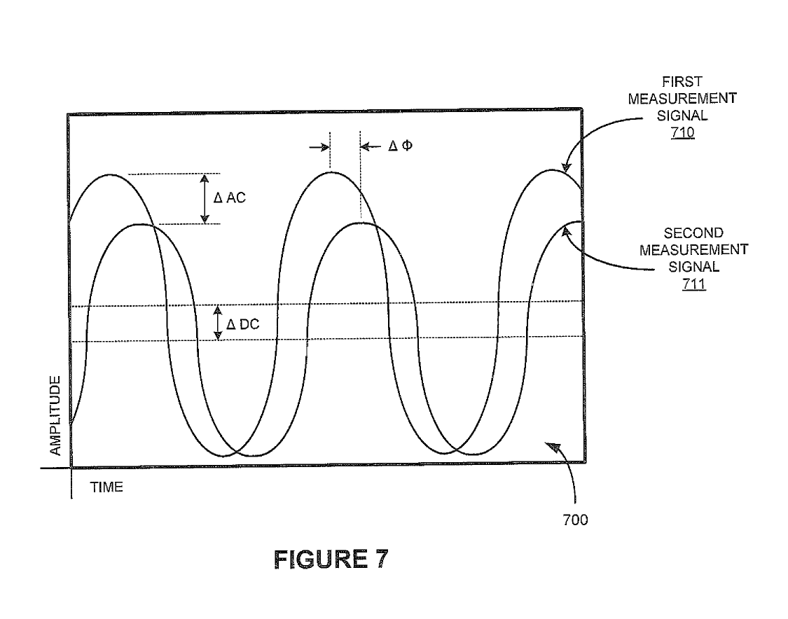

In this example embodiment, it has been determined that for many cases the maximum phase shift between any two channels is about 60 degrees of phase shift, and only a few cycles of the signals (illustrated in FIG. 7) are collected for each phase measurement performed. Sweep delay clock 620 provides a clock signal 681 to delay clock 630 which then provides control signal 682 to cross correlator 640. This delay clock 630 is used to delay one of the inputs in uniform time steps of a delta delay time as it is provided to cross correlator 640. This allows phase processor 650 to collect a plurality of cross correlation coefficients 683 from cross correlator 640, each coefficient 683 corresponding to an amount of delay added to one of the inputs.

In other words, phase processing module 600 operates by receiving two inputs, generating a plurality of delayed digital signals of one of the inputs, determining a cross correlation coefficient 683 for each of the plurality of delayed digital signals with respect to the non-delayed input, and calculating a phase delay between the two inputs based on the plurality of cross correlation coefficients 683.

A plurality of cross correlation coefficients 683 from cross correlator 640 are stored as a function of the delay clock time. These coefficients are obtained on both sides of a zero crossing of the cross correlation coefficients 683. Both positive and negative cross correlation coefficients 683 are determined. The zero crossing is interpolated from the best fit to a straight line of the coefficients 683 with respect to the delay clock time. The zero crossing point is at 90 degrees of phase shift of the two signals, which is the phase shift of the signals plus the delay clock time at the zero crossing point. Since the zero crossing point is known to represent 90 degrees of phase shift, the actual phase shift may be calculated by subtracting the phase shift represented by the delay clock time at the zero crossing from 90 degrees.

In other words, the actual phase shift may be calculated by:

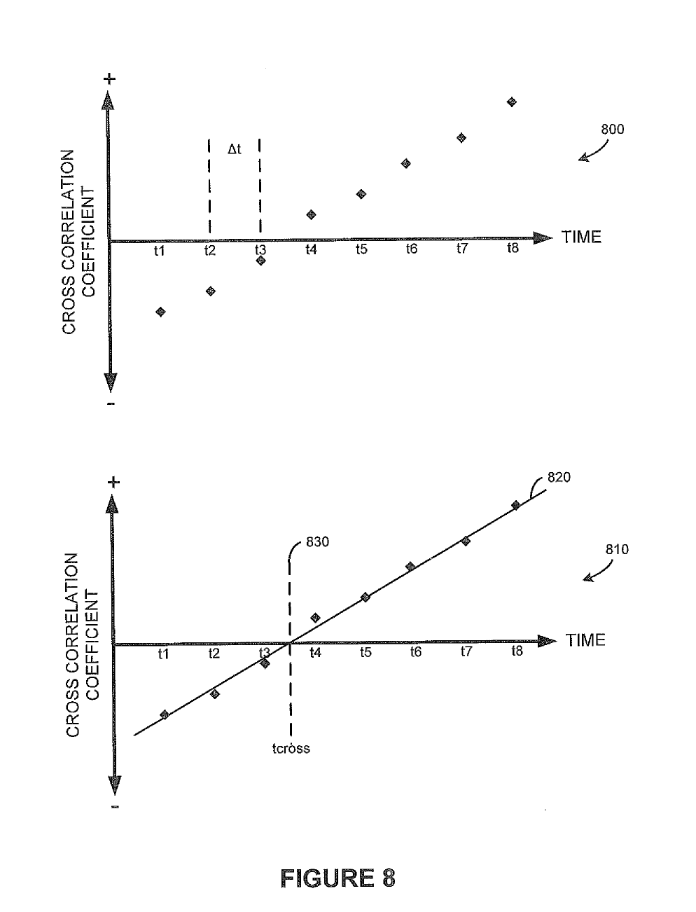

φ=90°−(Δt×f×360°)

where φ is the phase delay in degrees, Δt is the delay clock time in seconds, and f is the frequency of the signals in hertz. This calculation is illustrated in further detail in FIG. 8.

Note that the delta delay time must be small enough to enable calculation of the phase shift with a small degree of error. The allowable degree of error may be determined for each embodiment of the present invention. In order to get meaningful tissue information the phase differential between the two tissue signals typically should be accurate to better than about 0.01 degrees of phase. The phase resolution of the system is the clock speed of the delay clock relative to the intermediate frequency wavelength time. For example, if the IF frequency is 10 kHz then a sweep delay clock speed of 500 MHz provides a phase resolution of 0.0072 degrees of phase. This desired phase resolution is obtained when the sweep delay clock frequency is at least 50,000 times the intermediate frequency. Note that these determinations are based on an IF of 10 kHz and a desired phase resolution of 0.01 degrees. Other embodiments may use different frequencies and desired phase resolutions, resulting in very different requirements for the frequency of the sweep delay clock. These resulting sweep delay clock frequencies may be much slower in some embodiments, or much faster in other embodiments.

FIG. 7 includes graph 700 illustrating example parameter measurements. Graph 700 can represent a snapshot of the first and second measurement signals as received at receiver module 440. First measurement signal 710 comprises a magnified portion of the first measurement signal 432 as received at receiver module 440. Second measurement signal 711 comprises a magnified portion of the second measurement signal 433 as received at receiver module 440. The oscillations seen in graph 700 represent the high frequency modulation of the associated detected optical signals, and do not represent pulsatile perturbations.

As shown in graph 700, a delta in the peaks of first measurement signal 710 and second measurement signal 711 is indicated by Δ AC, or a dynamic differential in amplitude between first measurement signal 710 and second measurement signal 711. A delta in the average values of first measurement signal 710 and second measurement signal 711 is indicated by Δ DC, or a static differential in amplitude between first measurement signal 710 and second measurement signal 711. A delta in the phase of first measurement signal 710 and second measurement signal 711 is indicated by ΔΦ, or a dynamic differential in phase delay between first measurement signal 710 and second measurement signal 711. In one example embodiment for determining phase delay, first measurement signal 710 is used as a baseline, and the timewise shift in second measurement 711 from first measurement signal 710 is indicative of the phase delay. These various deltas can be determined by PA detector 332 or processing module 310, and processed to determine the physiological parameters as discussed herein.

FIG. 8 includes two graphs 800 and 810 illustrating an example phase delay calculation technique using cross correlation analysis for phase processing module 600. In this example embodiment sweep delay clock 620 has a frequency of 1/Δt. Eight cross correlation coefficients are collected and plotted with respect to delay time in graph 800. The first data point at time t1 represents the cross correlation coefficient when one of the input signals is delayed by Δt. The second data point at time t2 represents the cross correlation coefficient calculated when one of the input signals is delayed by 2Δt. The remaining data points are calculated at each step of time as determined by sweep delay clock 620.

Graph 810 illustrates best fit straight line 820 as applied to the data from graph 800. This line 820 is interpolated to determine a zero crossing time 830 identified as tcross in graph 810. Zero crossing time 830 is used as described above by phase processor 650 to calculate a phase delay between the two inputs 661 and 671 to phase processing module 600.

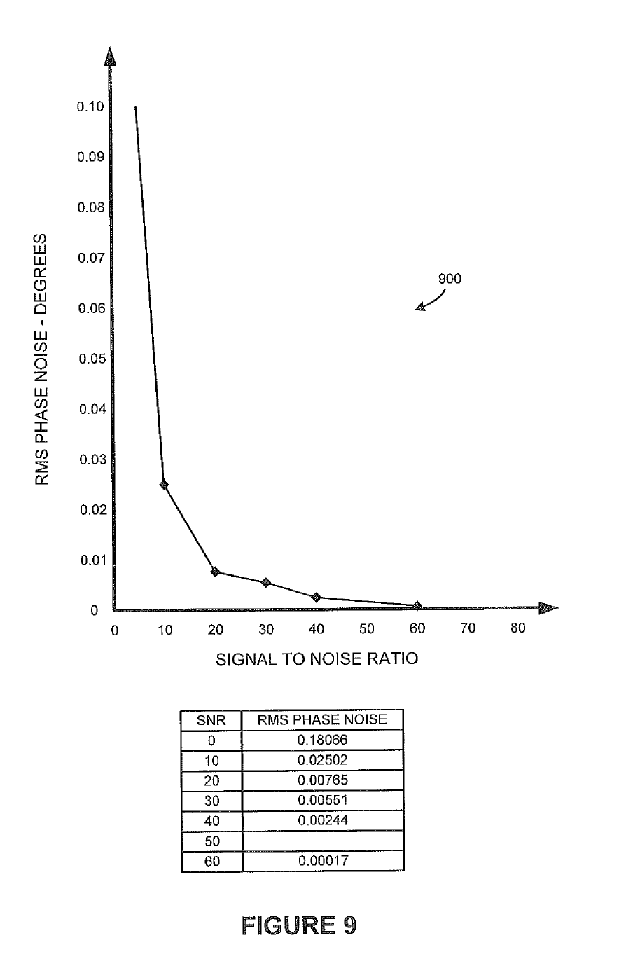

FIG. 9 includes a graph 900 illustrating example signal-to-noise ratios. The frequency of sweep delay clock 620 may be chosen to optimize signal-to-noise ratio (SNR) of the system, but it can be shown that a sample rate of 512 samples per wavelength of intermediate frequency with a 16 bit A/D converter provides sign limited SNR performance.

For the example embodiments discussed above, if a 10 millisecond sample rate per channel is desired, this rate equates to 3.3 milliseconds per channel, which for a 10 kHz intermediate frequency provides about 32 cycles of intermediate frequency if the total switching time is about 0.1 milliseconds. The ‘cycles’ as discussed herein can refer to a count of wavelength periods over time for a particular signal.

FIG. 9 shows expected performance based on simulations for the phase accuracy that may be obtained for 32 cycles of a signal for various signal to noise ratios of the signal, This simulation is based on using only the 85 degree and the 95 degree points around 90 degrees of phase shift. More accuracy may be obtained by using more computation points to make the interpolation. Graph 900 indicates that a SNR of about 20 dB should be adequate to achieve the desired phase accuracy using the approach described above. Note that the signal and frequencies shown in FIG. 9 are for illustrative purposes only, Other embodiments may have vastly different results than those illustrated in FIG. 9, which is simply a single example of one embodiment.

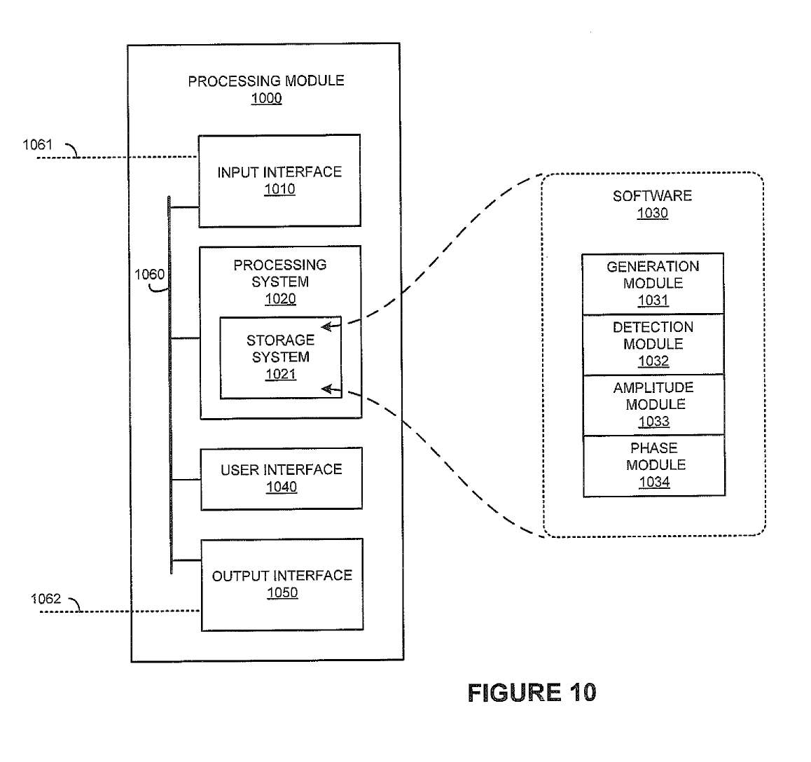

FIG. 10 is a block diagram illustrating processing module 1000, as an example of processing module 110 found in FIG. 1 or processing module 310 found in FIG. 3, although processing module 110 or processing module 310 can use other configurations. Processing module 1000 includes, input interface 1010, processing system 1020, user interface 1040, and output interface 1050. Input interface 1010, processing system 1020, user interface 1040, and output interface 1050 are shown to communicate over a common bus 1060 for illustrative purposes. It should be understood that discrete links can be employed, such as network links or other circuitry. Processing module 1000 may be distributed or consolidated among equipment or circuitry that together forms the elements of processing module 1000. In some examples, user interface 1040 is not included in processing module 1000.

Input interface 1010 comprises a communication interface for communicating with other circuitry and equipment, such as with receiver module 130, user interface 312, or ADC 334-335. Input interface 1010 can include transceiver equipment exchanging communications over the associated link 1061. It should be understood that input interface 1010 can include multiple interfaces, pins, transceivers, or other elements for communicating with multiple external devices. Input interface 1010 also receives command and control information and instructions from processing system 1020 or user interface 1040 for controlling the operations of input interface 1010. Link 1061 can use various protocols or communication formats as described herein for links 170-171, 340-344, or 350-356, including combinations, variations, or improvements thereof.

Processing system 1020 includes storage system 1021. Processing system 1020 retrieves and executes software 1030 from storage system 1021. In some examples, processing system 1020 is located within the same equipment in which input interface 1010, user interface 1040, or output interface 1050 are located. In further examples, processing system 1020 comprises specialized circuitry, and software 1030 or storage system 1021 can be included in the specialized circuitry to operate processing system 1020 as described herein. Storage system 1021 can include a non-transitory computer-readable medium such as a disk, tape, integrated circuit, server, flash memory, or some other memory device, and also may be distributed among multiple memory devices.

Software 1030 may include an operating system, logs, utilities, drivers, networking software, tables, databases, data structures, and other software typically loaded onto a computer system. Software 1030 can contain application programs, server software, firmware, processing algorithms, or some other form of computer-readable processing instructions. When executed by processing system 1020, software 1030 directs processing system 1020 to operate as described herein, such as instruct transmission modules on signal modulations, receive characteristics of PDWs, or process the characteristics of PDWs to determine blood parameters, among other operations.

In this example, software 1030 includes generation module 1031, detection module 1032, amplitude module 1033, and phase module 1034. It should be understood that a different configuration can be employed, and individual modules of software 1030 can be included in different equipment in processing module 1000. Generation module 1031 determines modulation parameters for use by a transmission module or signal synthesis circuitry, such as modulation frequency, phase delay for reference signals, laser activation periods, TDM, FDM, or CDM parameters, among other operations. Detection module 1032 receives receive characteristics of PDWs as detected by external circuitry, and processes the characteristics of the PDWs to determine blood parameters, among other operations. Detection module 1032 can receive reference signals from a transmission module or signal synthesis circuitry for processing with the characteristics of the PDWs.

Amplitude module 1033 receives measurement signals and determines amplitudes of the measurement signals. Phase module 1034 receives a pair of signals and determines the phase difference between the signals using a cross correlation method.

User interface 1040 includes equipment and circuitry to communicate information to a user of processing module 1000. Examples of the equipment to communicate information to the user can include displays, indicator lights, lamps, light-emitting diodes, haptic feedback devices, audible signal transducers, speakers, buzzers, alarms, vibration devices, or other indicator equipment, including combinations thereof. The information can include blood parameter information, waveforms, summarized blood parameter information, graphs, charts, processing status, or other information. User interface 1040 also includes equipment and circuitry for receiving user input and control, such as for beginning, halting, or changing a measurement process or a calibration process. Examples of the equipment and circuitry for receiving user input and control include push buttons, touch screens, selection knobs, dials, switches, actuators, keys, keyboards, pointer devices, microphones, transducers, potentiometers, non-contact sensing circuitry, or other human-interface equipment.

Output interface 1050 comprises a communication interface for communicating with other circuitry and equipment, such as with transmission module 120, signal synthesizer 320, or user interface 312. Output interface 1050 can include transceiver equipment exchanging communications over the associated link 1062. It should be understood that output interface 1050 can include multiple interfaces, pins, transceivers, or other elements for communicating with multiple external devices. Output interface 1050 also receives command and control information and instructions from processing system 1020 or user interface 1040 for controlling the operations of output interface 1050. Link 1062 can use various protocols or communication formats as described herein for links 170-171, 340-344, or 350-356, including combinations, variations, or improvements thereof.

Bus 1060 comprises a physical, logical, or virtual communication link, capable of communicating data, control signals, and communications, along with other information. In some examples, bus 1060 is encapsulated within the elements of processing module 1000, and may be a software or logical link. In other examples, bus 1060 uses various communication media, such as air, space, metal, optical fiber, or some other signal propagation path, including combinations thereof. Bus 1060 can be a direct link or might include various equipment, intermediate components, systems, and networks.

The included descriptions and drawings depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the invention. Those skilled in the art will also appreciate that the features described above can be combined in various ways to form multiple embodiments. As a result, the invention is not limited to the specific embodiments described above, but only by the claims and their equivalents.

What is claimed is:

1. A system to optically measure a physiological parameter of tissue of a patient comprising:
a tissue interface assembly configured to emit an optical signal into the tissue, receive a first measurement signal based on the optical signal propagating along a first path, receive a second measurement signal based on the optical signal propagating along a second path, and transfer the first measurement signal and the second measurement signal for delivery to a processing system; and
the processing system coupled to the tissue interface assembly and configured to receive the first measurement signal and the second measurement signal, determine a phase delay between the first measurement signal and the second measurement signal based on a cross correlation analysis, and identify a value of the physiological parameter of the patient based on at least the phase delay between the first measurement signal and the second measurement signal.

2. The system of claim 1, wherein the processing system comprises:
a receiver configured to convert the first measurement signal and the second measurement signal into a first digital signal and a second digital signal;
a cross correlator configured to process the first digital signal and the second digital signal in a cross correlation analysis to determine a phase delay between the first digital signal and the second digital signal; and
a phase processor configured to process the phase delay between the first digital signal and the second digital signal to identify the physiological parameter of the patient.

3. The system of claim 2, wherein the cross correlator is configured to:
create a plurality of delayed second digital signals, each having a delay time, by delaying the second digital signal by multiples of a delta delay time;
process the first digital signal with the plurality of delayed second digital signals to produce cross correlation coefficients between the first digital signal and a delayed second digital signal for each delay time;
calculate a best fit straight line for the cross correlation coefficients versus the delay times;
calculate a zero coefficient delay time by interpolating the best fit straight line to determine a time at which the cross correlation coefficients are equal to zero; and
calculate the phase delay between the first digital signal and the second digital signal from the zero coefficient delay time.

4. The system of claim 3, wherein the phase delay between the first digital signal and the second digital signal is calculated based on at least the zero coefficient delay time and a frequency of the first digital signal or the second digital signal.

5. The system of claim 3, wherein the processing system comprises:
a sweep delay clock configured to generate a clock signal having a clock frequency of 1/(the delta delay time), wherein the clock signal is used to create the plurality of delayed second digital signals, by delaying the second digital signal by integral multiples of the delta delay time.

6. The system of claim 5, wherein the clock frequency is selected to provide a phase resolution of less than 0.01 degrees of phase.

7. The system of claim 5, wherein the optical signal has an intermediate frequency, and the clock frequency is at least 50,000 times the intermediate frequency.

8. The system of claim 3, wherein the processing system further comprises:
a first low pass filter configured to remove components of the first measurement signal having frequencies above a first cutoff frequency before the first measurement signal is converted into the first digital signal; and
a second low pass filter configured to remove components of the second measurement signal having frequencies above a second cutoff frequency before the second measurement signal is converted into the second digital signal.

9. A system to optically measure a physiological parameter of tissue of a patient comprising:
a transmission module, configured to generate an optical signal;
a tissue interface assembly coupled to the transmission module and configured to receive the optical signal, emit the optical signal into the tissue, receive a reference signal based on the optical signal propagating along a first path, receive a measurement signal based on the optical signal propagating along a second path, and transfer the reference signal and the measurement signal for delivery to a receiver module;
the receiver module coupled to the tissue interface assembly and configured to receive the reference signal and the measurement signal from the tissue interface assembly, convert the reference signal into a digital reference signal, and convert the measurement signal into a digital measurement signal; and
a back end module coupled to the receiver module and configured to receive the digital reference signal and the digital measurement signal from the receiver module, determine a phase delay between the digital reference signal and the digital measurement signal based on a cross correlation analysis, and identify a value of the physiological parameter of the patient based on at least the phase delay between the digital reference signal and the digital measurement signal.

10. The system of claim 9, wherein the receiver module comprises:
a reference low pass filter configured to remove components of the reference signal having frequencies above a reference cutoff frequency before the reference signal is converted into the digital reference signal; and
a measurement low pass filter configured to remove components of the measurement signal having frequencies above a measurement cutoff frequency before the measurement signal is converted into the digital measurement signal.

11. The system of claim 10, wherein the receiver module further comprises:
a reference analog-to-digital convertor configured to convert the reference signal into the digital reference signal; and
a measurement analog-to-digital convertor configured to convert the measurement signal into the digital measurement signal.

12. The system of claim 9, wherein the back end module comprises a cross correlator, and the cross correlator is configured to:
create a plurality of delayed digital measurement signals, each having a delay time, by delaying the digital measurement signal by multiples of a delta delay time;
process the digital reference signal with the plurality of delayed digital measurement signals to produce cross correlation coefficients between the digital reference signal and a delayed digital measurement signal for each delay time;
calculate a best fit straight line for the cross correlation coefficients versus the delay times;
calculate a zero coefficient delay time by interpolating the best fit straight line to determine a time at which the cross correlation coefficients are equal to zero; and
calculate a phase delay between the digital reference signal and the digital measurement signal from the zero coefficient delay time.

13. The system of claim 12, wherein the phase delay between the digital reference signal and the digital measurement signal is calculated based on at least the zero coefficient delay time and a frequency of digital reference signal.

14. The system of claim 12, wherein the cross correlator comprises:
a sweep delay clock configured to generate a clock signal having a clock frequency of 1/(the delta delay time), wherein the clock signal is used to create the plurality of delayed digital measurement signals, by delaying the digital measurement signal by integral multiples of the delta delay time.

15. The system of claim 14, wherein the clock frequency is configured to provide a phase resolution of less than 0.01 degrees of phase.

16. The system of claim 14, wherein the optical signal has an intermediate frequency, and the clock frequency is at least 50,000 times the intermediate frequency.

17. A method to optically measure a physiological parameter of tissue of a patient comprising:
emitting an optical signal into the tissue;
receiving a first measurement signal based on the optical signal propagating along a first path;
receiving a second measurement signal based on the optical signal propagating along a second path;
determining a phase delay between the first measurement signal and the second measurement signal based on a cross correlation analysis; and
identifying a value of the physiological parameter of the patient based on at least the phase delay between the first measurement signal and the second measurement signal.

18. The method of claim 17, further comprising:
converting the first measurement signal into a first digital signal;
converting the second measurement signal into a second digital signal;
determining a phase delay between the first digital signal and the second digital signal based on a cross correlation analysis; and
identifying a value of the physiological parameter of the patient based on at least the phase delay between the first digital signal and the second digital signal.

19. The method of claim 18, further comprising:
creating a plurality of delayed second digital signals, each having a delay time, by delaying the second digital signal by multiples of a delta delay time;
processing the first digital signal with the plurality of delayed second digital signals to produce cross correlation coefficients between the first digital signal and a delayed second digital signal for each delay time;
calculating a best fit straight line for the cross correlation coefficients versus the delay times;
calculating a zero coefficient delay time by interpolating the best fit straight line to determine a time at which the cross correlation coefficients are equal to zero; and
calculating the phase delay between the first digital signal and the second digital signal from at least the zero coefficient delay time and a frequency of the first digital signal or a frequency of the second digital signal.

20. The method of claim 19, further comprising:
generating a clock signal having a clock frequency of 1/(the delta delay tune), wherein the clock signal is used to create the plurality of delayed second digital signals, by delaying the second digital signal by integral multiples of the delta delay time.

21. A non-transitory computer-readable medium having instructions stored thereon for analyzing physiological parameters of patients, wherein the instructions, when executed by a processing system, direct the processing system to at least:
determine a phase delay based on a cross correlation analysis between a first measurement signal from an optical signal propagating along a first path through tissue in a patient and a second measurement signal from the optical signal propagating along a second path through the tissue; and
identify a value of the physiological parameter of the patient based on at least the phase delay between the first measurement signal and the second measurement signal.
